(19) United States
(12) Patent Application Publication
(10) Pub. No.: US 2010/0056754 A1
(43) Pub. Date: Mar. 4, 2010

(54) NANO-PUMP USING MOLECULAR MOTOR

(76) Inventor: Dong June Ahn, Seoul (KR)

Correspondence Address: EDWARDS ANGELL PALMER & DODGE LLP, P.O. BOX 55874, BOSTON, MA, 02205, US

(21) Appl. No.: 12/200,888

(22) Filed: Aug. 28, 2008

(51) Int. Cl. C07K 2/00; C07C 245/08

(52) U.S. Cl. 530/300; 534/843

(57) ABSTRACT

Nano-pumps including nano-motors that move dynamically in response to exterior stimuli, for example in response to photonic energy, electrical energy, a magnetic field, and/or a chemical concentration. The motion of such a molecular sized nano-motor structure can be managed and controlled so as to act as a pump. Such pumps could be used in nanobots in order to perform work.

### TECHNICAL FIELD

The present application relates generally to nano-technology structure and methods.

### BACKGROUND

Molecular size motors, also referred to herein as nano-motors, refer to molecule sized structures that move dynamically in a nano to micrometer scale. The ability to control and manage the motion of such molecular-motors would provide a platform for building nano-pumps and nano-engines employing such motors.

### BRIEF SUMMARY

According to one embodiment, a nano-motor is provided. Such a motor includes a stimulus receptor and a molecular piston member attached to the stimulus receptor. The stimulus-receptor is responsive to one or more external stimuli so as to selectively move the molecular piston member in response to the stimulus. Movement of the molecular piston member of such a nano-motor can be used within a larger system (e.g., a nano-pump).

According to one embodiment, a nano-pump system is provided. Such a system includes an enclosing body member (e.g., for containing a fluid to be moved or compressed) and a molecular motor structure including a molecular piston member. The molecular-motor (i.e., a nano-motor) is responsive to one or more external stimuli so as to move the molecular piston member within the enclosing body member in response to the stimulus. Movement of the molecular piston member of such a nano-pump can be used to move or compress a fluid within the enclosure.

According to one embodiment, the stimulus which causes the molecular-motor to selectively move comprises one or more of electromagnetic (e.g., photonic) radiative energy, electrical energy, a magnetic field, or a chemical concentration (e.g., which results in a chemical and/or biochemical reaction that effects movement of the structure). In such a way, the motion of such a molecular sized structure can be managed and controlled so as to act as part of a pump system. Such pumps could be used in nanobots in order to perform work.

For example, in one method of use, a nano-pump system including an enclosing body member and a molecular-motor structure responsive to an external stimulus as described above is provided. The molecular-motor structure is exposed to a selected stimulus, resulting in movement of the molecular-piston member of the motor structure.

This summary is provided to introduce a selection of concepts in a simplified form that are further described below in the Detailed Description. This summary is not intended to identify key features or essential characteristics of the claimed subject matter, nor is it intended to be used as an aid in determining the scope of the claimed subject matter.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1A is a schematic representation of an illustrative molecular-motor including a piston member in the shape of a nanorod.

FIG. 1B is a schematic representation of another illustrative molecular-motor including a piston member in the shape of a nanorod.

FIG. 2A is a schematic representation of an illustrative nano-pump system including a molecular-motor similar to that of FIG. 1A disposed within an enclosing body that acts as a container for a fluid.

FIG. 2B is a schematic representation of the nano-pump system of FIG. 2A, in which the molecular-motor has moved to a different position in response to a stimulus, resulting in movement and/or compression of the fluid within the enclosing body.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein.

According to one embodiment, a nano-motor is provided. Such a motor includes a stimulus receptor and a molecular piston member attached to the stimulus receptor. The stimulus-receptor is responsive to one or more external stimuli so as to selectively move the molecular piston member in response to the stimulus. Movement of the molecular piston member of such a nano-motor can be used within a larger system (e.g., a nano-pump).

According to another embodiment, a nano-pump system is provided. Such a system includes an enclosing body member (e.g., for containing a fluid to be moved or compressed) and a molecular motor structure including a molecular piston member. The molecular-motor structure (i.e., a nano-motor) is responsive to one or more external stimuli so as to move the molecular piston member within the enclosing body member in response to the stimulus. Movement of the molecular piston member of such a nano-pump can be used to move or compress a fluid within the enclosure. Such movement can be used to achieve desired motion or work within a larger system (e.g., a nanobot).

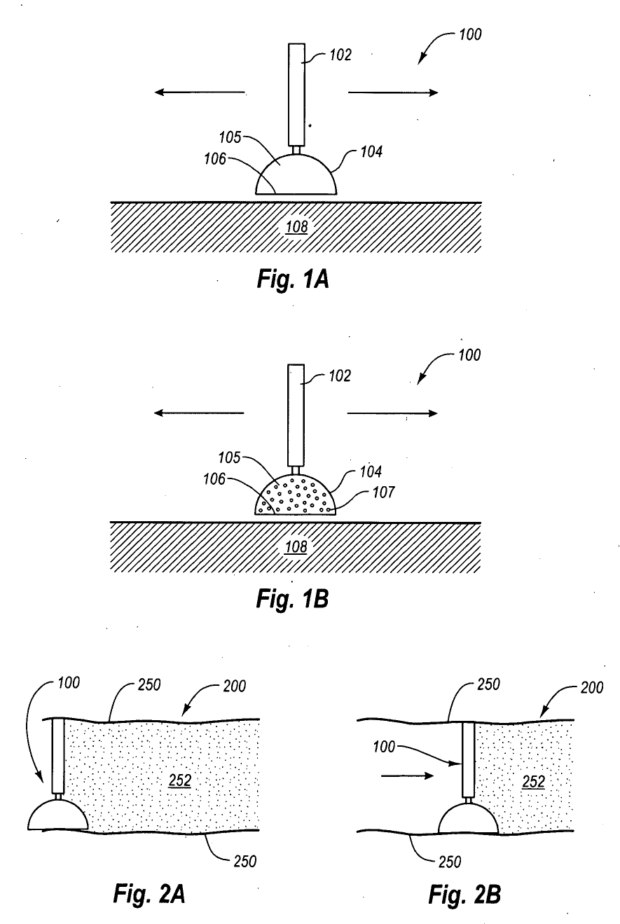

FIG. 1A illustrates a schematic representation of a molecular-motor 100. As illustrated in FIG. 1A, molecular-motor 100 includes a molecular piston member 102 and a stimulus receptor 104. Although the schematic illustration shows molecular piston member 102 having a generally rectangular shape (i.e., a nano-rod) and stimulus receptor having a generally hemispherical shape, the molecular motor structure 100 and its subcomponents may be of any shape. By way of illustration, the nano-rod or other shaped molecular-piston member may have a length in a broad range between about 10 nm and about 100,000 nm, in an intermediate range between about 50 nm and about 10,000 nm, and in a narrow range between about 100 nm and about 1000 nm.

Molecular piston member 102 and stimulus receptor 104 may be formed of a carbon based material, for example an organic carbon backbone structure. One or more of the structures may comprise a cyclophane structure, formed of large molecular rings. In another embodiment, the molecular piston member 102 and/or stimulus receptor 104 may comprise a protein structure. For example, lower surface 106 of stimulus receptor 104 may comprise actin protein, while adjacent surface 108 may comprise myosin protein. Upon exposure to adenosine tri-phosphate (ATP), the myosin protein surface 108 alters shape as it engages with the adjacent actin lower surface 106, pulling the actin stimulus receptor (and attached molecular piston member 102) along the myosin protein surface 108. Exposure to additional ATP (and its subsequent decomposition to ADP and inorganic phosphate) results in further movement of the myosin stimulus receptor along the actin protein surface.

Stimulus receptor 104 is responsive to one or more stimuli which effect movement of the molecular motor structure. Examples of such external stimuli which may result in movement of molecular-motor 100 include, but are not limited to, exposure to photonic energy (e.g., electromagnetic radiation of a particular wavelength), electrical energy (e.g., application of a particular voltage and/or current), a magnetic field (e.g., of a particular strength), or a chemical concentration. For example, exposure to a particular concentration of one or more given chemicals may result in a redox or other chemical or biochemical reaction which effects movement of stimulus receptor 104 and molecular piston member 102 (i.e., as piston member 102 is attached to receptor 104, movement of receptor 104 results in movement of piston member 102). For example, ATP is one example of a chemical whose concentration may result in movement of stimulus receptor 104. ATP may be supplied to a myosin surface 108 by an external solution, as shown in FIG. 1A. Upon exposure to a concentration of ATP, the myosin protein structure alters shape, engages the adjacent actin surface, and pulls the actin structure (with any attached molecular-piston member) along the myosin surface. In another embodiment (FIG. 1B), a quantity of ATP 107 may be stored within or otherwise associated with stimulus receptor 104. Stimulus receptor 104 may include a molecular-valve (e.g., an azobenzene cis-trans switch) which acts to open chamber 105, releasing ATP stored therein upon application of a stimulus (e.g., light in the case of an azobenzene switch). Additional details regarding molecular-valves are described in Nguyen, Thoi D. et al., A Reversible Molecular Valve, Proceedings of the National Academy of Sciences of the United States of America Vol. 102 No. 29 (Jul. 19, 2005) pp. 10029-10034; also Browne, Wesley R. et al. Making Molecular Machines Work, Vol. 1 October 2006, pp. 25-35; as well as a United States Patent application bearing attorney docket number 17655.6 and filed the same day as the present application. Each of the above articles and patent application are herein incorporated by reference. In an alternative embodiment, the myosin/actin designations may be opposite those illustrated (i.e., lower surface 106 of stimulus receptor 104 may comprise myosin and adjacent surface 108 may comprise actin).

In one embodiment, the motor may be reversible and reusable, capable of movement in one direction and then back again. Such movements may be controlled through the stimulus provided. For example, one wavelength of light or chemical concentration may result in movement of the molecular motor in one direction, while a different wavelength of light or another chemical concentration may result in movement in an opposite direction, moving the molecular-motor back to its original location. Similar results may be achieved with different types of stimuli (e.g., electrical and/or magnetic) or with combinations of different types of stimuli (e.g., one type of stimuli may result in a first defined movement of the molecular-motor, while the movement may be reversed by a second movement achieved through a different stimulus mechanism).

For example, additional information regarding organic molecules including portions that can be caused to move upon application of photonic, electrical, magnetic, or chemical stimuli, their manufacture, use and properties, is found in Nguyen, Thoi D. et al., A Reversible Molecular Valve, Proceedings of the National Academy of Sciences of the United States of America Vol. 102 No. 29 (Jul. 19, 2005) pp. 10029-10034, and also Browne, Wesley R. et al. Making Molecular Machines Work, Vol. 1 October 2006, pp. 25-35, the disclosures of which are incorporated herein by reference. For example, the Nguyen article describes a rotaxane cyclic molecule, which is an example of a cyclophane. The rotaxane molecule includes a ring portion that moves from one attachment location to another upon addition of Fe(ClO₄)₃. Such geometric rearrangement of the molecule's structure can be exploited to operate as a molecular-motor. Movement and reattachment of the ring portion to a different location relative to the molecule effected by addition of Fe(ClO₄)₃ can be reversed by subsequent addition of a weak acid (e.g., ascorbic acid).

Another example of an illustrative stimulus receptor may include an azobenzene structure bonded to a molecular-piston member (e.g., an aliphatic, cyclophane, or protein structure). An example of an azobenzene structure that may be included within a nano-motor is illustrated below:

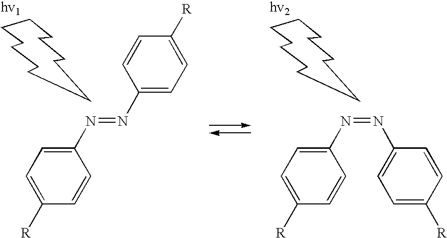

In the illustration, the R groups may represent any relatively bulky group to which the benzene rings are attached (e.g., an aliphatic, cyclophane, and/or protein portion). In addition, it is not necessary that the R groups be identical. A cis to trans photo-isomerization of the azobenzene structure can result in movement of the bulky portions (e.g., a molecular-piston member) of the molecule from one side of the molecule to the other side of the molecule. The stimulus in such an example is exposure to a first wavelength of light to convert from a cis to trans configuration, followed by exposure to a second, different wavelength of light to reverse back to the original configuration.

In one example, stimulus receptor includes a lower surface 106. In FIG. 1A, surface 106 is illustrated as opposite and adjacent to a substrate surface 108. For example, in an embodiment where movement is achieved through the presence of a chemical concentration, one or more chemicals present in a desired concentration may be involved in a chemical or biochemical surface reaction that occurs at the interface between surface 106 and surface 108. The primary result of the surface reaction is movement of stimulus receptor 104 (and attached molecular piston member 102) along and relative to surface 108.

For example, the stimulus receptor may include a metal capable of catalyzing decomposition of hydrogen peroxide (which acts as a chemical fuel). Decomposition of the hydrogen peroxide into water and O₂ results in generation of a concentration gradient and/or difference in surface tension (when in a solution environment), providing the stimulus to move the stimulus receptor 104 and its attached molecular piston member 102. Such movement induced by a chemical concentration gradient may be similar to that used by some bacteria for movement.

In another embodiment, an illustrative molecular-motor may comprise a protein structure including an internal channel surrounded by the protein structure. Such an internal channel may be filled with a fluid to be moved. Upon exposure to a stimulus (e.g., a particular wavelength of light), the protein changes shape so that the channel collapses, pushing the fluid outwardly relative to the channel. Upon exposure to another stimulus (e.g., a different wavelength of light), the protein structure expands to its original configuration, restoring the channel.

For example, an illustrative molecular-motor may comprise a protein structure including an internal channel surrounded by the protein structure. One such channel protein is described in the Browne article. The channel protein of the Browne article comprises a channel protein modified with a photochemical active spiropyran switch, as illustrated below.

The reversible switch acts as a valve control for a 3 nm channel. The channel can be opened and closed upon exposure to ultraviolet and visible light, respectively. This is possible as the neutral switch molecule converts to a highly polar zwitterionic form upon exposure to ultraviolet light. Such a molecular-motor structure may form part of a larger nanobot structure in which the movement of the fluid within the channel is used to perform work or simply to effect movement of the larger structure (e.g., the force generated by expulsion of the fluid could be used to propel the larger nanobot structure through a fluid medium).

As illustrated in FIG. 2A, molecular-motor 100 may form part of a larger nano-pump system 200. Nano-pump system 200 includes a molecular-motor 100 disposed within an enclosing body member 250 (e.g., formed of a carbon based material, glass, silica, latex, polystyrene, carbon, silver, copper, other metal, or magnetic material). As illustrated, enclosure 250 is filled with a fluid 252 (e.g., water, an aqueous solution, an oil, air, or another gas), which is acted upon by molecular-motor 100. As seen in FIG. 2A, molecular-motor 100 is in a first position, and through application of a stimulus as described above and as shown in FIG. 2B, molecular-motor 100 moves further within enclosing body member 250. Because molecular-motor 100 fills substantially the full height of enclosing body member 250, it effectively forms a seal at the top (between molecular piston member 102 and enclosure 250) and bottom edges (between stimulus receptor 104 and enclosure 250) of enclosure 250, such that its movement from the first position as shown in FIG. 2A to the second position shown in FIG. 2B forces fluid 252 in that same direction of movement (or compresses the fluid—e.g., depending on whether the unseen opposite end of enclosure 250 is open or closed). Movement and/or compression of fluid 252 may be used to perform work within a larger nanobot.

By way of illustration, the enclosing body member may have a diameter in a broad range between about 100 nm and about 100,000 nm, in an intermediate range between about 500 nm and about 50,000 nm, and in a narrow range between about 1,000 nm and about 10,000 nm.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.”

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A nano-motor comprising:
a stimulus receptor; and
a molecular piston member attached to the stimulus receptor;
wherein the stimulus receptor is responsive to an exterior stimulus so as to selectively move the molecular piston member in response to an applied stimulus.

2. A nano-motor as recited in claim 1, wherein the molecular piston member comprises a nano-rod.

3. A nano-motor as recited in claim 1, wherein the stimulus receptor is activated by one or more stimuli selected from the group consisting of photonic energy, electrical energy, a magnetic field, and a chemical concentration.

4. A nano-motor as recited in claim 1, wherein the stimulus receptor is activated by exposure to a chemical concentration which results in a redox chemical reaction which results in movement of the molecular piston member.

5. A nano-motor as recited in claim 1, wherein the stimulus receptor is activated by exposure to a chemical concentration which results in a bio-chemical reaction which effects movement of the molecular piston member.

6. A nano-motor as recited in claim 5, wherein the biochemical reaction occurs at an interface between the stimulus receptor and an adjacent surface.

7. A nano-pump system comprising:
an enclosing body member; and
a molecular motor structure including a molecular piston member responsive to an exterior stimulus so as to selectively move the molecular piston member within the enclosing body member in response to an applied stimulus.

8. A nano-pump system as recited in claim 7, wherein the molecular piston member is activated by one or more stimuli selected from the group consisting of photonic energy, electrical energy, a magnetic field, and a chemical concentration.

9. A nano-pump system as recited in claim 7, wherein the molecular piston member is activated by exposure to a chemical concentration which results in a redox chemical reaction which effects movement of the molecular piston member.

10. A nano-pump system as recited in claim 7, wherein the molecular piston member is activated by exposure to a chemical concentration which results in a biochemical reaction which effects movement of the molecular piston member.

11. A nano-pump system as recited in claim 7, wherein the molecular piston member comprises a nano-rod.

12. A nano-pump system as recited in claim 11, wherein the nano-rod has a length between about 10 nm and about 100,000 nm.

13. A nano-pump system as recited in claim 7, wherein the enclosing body member has a diameter between about 100 nm and about 100,000 nm.

14. A method of using a nano-pump system comprising:
providing a nano-pump system comprised of:
an enclosing body member; and
a molecular motor structure including a molecular piston member responsive to an exterior stimulus so as to selectively move the molecular piston member within the enclosing body member in response to the stimulus; and
exposing the molecular-motor structure of the nano-pump system to an exterior stimulus configured to selectively move the molecular-piston member.

15. A method as recited in claim 14, wherein the molecular-motor structure is activated by one or more stimuli selected from the group consisting of photonic energy, electrical energy, a magnetic field, and a chemical concentration.

16. A method as recited in claim 14, wherein the molecular-motor structure is activated by exposure to a chemical concentration which results in a redox chemical reaction which results in movement of the molecular piston member.

17. A method as recited in claim 14, wherein the molecular piston member is activated by exposure to a chemical concentration which results in a biochemical reaction which effects movement of the molecular piston member.

18. A method as recited in claim 14, wherein movement of the molecular piston member is used to perform work.

19. A method as recited in claim 14, wherein movement of the molecular piston member is used to move a fluid contained within the enclosing body member.

20. A method as recited in claim 14, wherein movement of the molecular piston member is used to compress a fluid contained within the enclosing body member.
